(19) United States
(12) Patent Application Publication
(10) Pub. No.: US 2022/0257905 A1
(43) Pub. Date: Aug. 18, 2022

(54) CATHETER TRACTION DEVICES, SYSTEMS, AND METHODS

(71) Applicant: Becton, Dickinson and Company, Franklin Lakes, NJ (US)

(72) Inventors: John Lackey, West Valley City, UT (US); Megan Scherich, Salt Lake City, UT (US); Benjamin Hopwood, Salt Lake City, UT (US); Weston F. Harding, Lehi, UT (US); Curtis H. Blanchard, Riverton, UT (US); Jonathan Karl Burkholz, Salt Lake City, UT (US); Yiping Ma, Layton, UT (US); Justin G. Hortin, Farmington, UT (US)

(21) Appl. No.: 17/673,463

(22) Filed: Feb. 16, 2022

Related U.S. Application Data
(60) Provisional application No. 63/149,988, filed on Feb. 16, 2021.

(51) Int. Cl. A61M 25/01

(52) U.S. Cl. CPC A61M 25/0147; A61M 25/0606; A61M 25/0136

(57) ABSTRACT

A catheter traction system to apply traction to an intravenous catheter. The catheter traction system may include a rotary device and an extension member. The extension member may extend from the rotary device and be configured to couple to a catheter assembly. In response to activation of the rotary device, the extension member may be configured to apply traction to the catheter assembly.

### CROSS-REFERENCE TO RELATED APPLICATION

The present application claims priority to U.S. Provisional Application Ser. No. 63/149,988 entitled “Catheter Traction Devices, Systems, and Methods”, filed Feb. 16, 2021, the entire disclosure of which is hereby incorporated by reference in its entirety.

### BACKGROUND

Catheters are commonly used to infuse fluids into vasculature of a patient. For example, catheters may be used for infusing normal saline solution, various medicaments, or total parenteral nutrition. Catheters may also be used for withdrawing blood from the patient.

A catheter may include an over-the-needle peripheral intravenous (“IV”) catheter. In this case, the catheter may be mounted over an introducer needle having a sharp distal tip. The catheter and the introducer needle may be assembled so that the distal tip of the introducer needle extends beyond the distal tip of the catheter with the bevel of the needle facing up away from skin of the patient. The catheter and introducer needle are generally inserted at a shallow angle through the skin into vasculature of the patient.

In order to verify proper placement of the introducer needle and/or the catheter in the blood vessel, a clinician generally confirms that there is “flashback” of blood in a flashback chamber. After placement of the introducer needle has been confirmed, the clinician may remove the introducer needle, leaving the catheter in place for future blood withdrawal or fluid infusion.

Overtime the catheter can become occluded at a tip of the catheter due to presence of fibrin sheath, thrombus, or vein walls or valves. Occlusions can limit functionality of the catheter for infusion and/or blood draw. The subject matter claimed herein is not limited to embodiments that solve any disadvantages or that operate only in environments such as those described above. Rather, this background is only provided to illustrate one example technology area where some implementations described herein may be practiced.

### SUMMARY OF THE INVENTION

The present disclosure relates generally to catheter traction devices, systems, and methods to facilitate application of traction to a catheter. In some instances, a catheter, such as a peripheral intravenous catheter, midline catheter, or peripherally inserted central catheter, can become occluded at a tip of the catheter due to presence of fibrin sheath, thrombus, or vein walls or valves. Occlusions can limit functionality of the catheter for infusion and/or blood draw. In some embodiments, traction may be applied to the tip of the catheter to re-position the tip within the vein, which may reduce occlusion and facilitate blood draw success. In some embodiments, applying traction may include pulling the catheter in a proximal direction. In some embodiments, traction may be applied to the catheter by movement of a catheter assembly. In some embodiments, a catheter traction system may use rotational movement to apply traction to the catheter tip, thereby facilitating blood draws and infusions by opening a fluid path through the catheter.

In some embodiments, the catheter traction system to apply traction to an intravenous catheter may include a rotary device. In some embodiments, the catheter traction system may include an extension member, which may extend from the rotary device and may be configured to couple to the catheter assembly. In some embodiments, in response to activation of the rotary device, the extension member may be configured to apply traction to the catheter assembly. In some embodiments, the extension member may be rigid. In other embodiments, the extension member may be flexible.

The extension member may be coupled to the catheter assembly in various ways. In some embodiments, the catheter traction system may include a spring hose clamp disposed at a distal end of the extension member and configured to couple to the catheter assembly. In some embodiments, the catheter traction system may include a connector disposed at a distal end of the extension member, and the connector may include a housing, a button depressible into the housing, and a spring disposed between an inner surface of the housing and the button. In some embodiments, the button may include an arm and the housing may include another arm. In some embodiments, the arm and the other arm may form a mouth configured to grasp the catheter assembly. In some embodiments, the spring may bias the button in a first position in which the mouth is closed, facilitating coupling to the catheter assembly. In some embodiments, depression of the button from the first position to a second position may open the mouth, facilitating uncoupling of the connector from the catheter assembly. In some embodiments, the catheter traction system may include a connector disposed at a distal end of the extension member and configured to snap onto the catheter assembly.

In a first set of embodiments, the rotary device may include a base, a groove, and a rotary element. In some embodiments, the rotary element may include a peg. In some embodiments, the rotary element may be configured to rotate with respect to the base. In some embodiments, in response to the peg being forced into the groove, rotation of the rotary element with respect to the base may be reduced. In some embodiments, in response to rotation of the rotary element with respect to the base, the extension member may be configured to apply traction to the catheter assembly.

In some embodiments, the groove may be disposed within the base. In some embodiments, the groove may be circular. In some embodiments, the groove may be c-shaped. In some embodiments, the peg may be disposed within the groove. In some embodiments, an outer portion of the groove may be narrower than an inner portion of the groove to create an interference fit with the peg in response to the peg being forced into the groove.

In some embodiments, the groove may be disposed within the base, and the rotary element may include a disc-shaped body. In some embodiments, the peg may extend downwardly from a bottom of the disc-shaped body.

In some embodiments, the rotary device may include a lid covering the base. In some embodiments, the groove may be disposed within the lid. In some embodiments, the rotary element may include the disc-shaped body, and the peg may extend upwardly from a top of the disc-shaped body. In some embodiments, the rotary device may include the lid, which may be coupled to the base, and the rotary element may include a neck portion extending through the lid. In some embodiments, the lid may hold the peg in the groove.

In some embodiments, the groove may include one or more notches. In some embodiments, in response to the peg being forced into the notches, rotation of the rotary element with respect to the base may be reduced.

In a second set of embodiments, a rotary device may include a base, which may include an opening, a cutout, and a compliant portion. In some embodiments, the opening may be spaced apart from the cutout by the compliant portion. In some embodiments, the compliant portion may include a first shape. In some embodiments, the rotary element may be disposed within the opening and configured to rotate with respect to the base. In some embodiments, an outer edge of the rotary element may include a second shape complementary to the first shape and configured to fit into the first shape. In some embodiments, in response to the second shape being disposed within the first shape, rotation of the rotary element with respect to the base may be reduced. In some embodiments, in response to rotation of the rotary element with respect to the base, the extension member may be configured to apply traction to the catheter assembly.

In a third set of embodiments, the rotary device may include a base, which may include a shaft. In some embodiments, an outer surface of the shaft may include multiple teeth. In some embodiments, the rotary device may include a rotary element, which may include a slot. In some embodiments, the rotary device may include a tab, which may be disposed within the slot of the rotary element. In some embodiments, the tab may include an opening, and the shaft extends through the opening. In some embodiments, an edge of the opening may include multiple other teeth.

In some embodiments, the rotary device may include a spring, which may be disposed within the slot between an inner surface of the rotary element and the tab. In some embodiments, the spring may bias the tab in a first position. In some embodiments, in response to the tab being in the first position, the plurality of teeth may be engaged with the other teeth. In some embodiments, in response to compression of the spring and the tab being in a second position, the teeth may not be engaged with the other teeth and the rotary element may be configured to rotate with respect to the base. In some embodiments, in response to rotation of the rotary element with respect to the base, the extension member may be configured to apply traction to the catheter assembly.

In a fourth set of embodiments, the rotary device may include a ratchet, which may include a gear and a pawl. In some embodiments, the extension member may be coupled to the ratchet. In some embodiments, in response to rotation of the gear, the extension member may be configured to apply traction to the catheter assembly.

In some embodiments, the rotary device may include a base, and the gear and the pawl may be mounted to the base. In some embodiments, the rotary device may include a handle, which may extend upwardly from the gear. In some embodiments, the extension member may be wrapped around the handle.

In a fifth set of embodiments, the rotary device may include a rotary element configured to rotate about an axis between multiple preset locations. In some embodiments, the rotary device may include a connector disposed at a distal end of the extension member. In some embodiments, the connector may be configured to couple to the catheter assembly. In some embodiments, the rotary element may include a pivot point disposed between the distal end of the extension member and the connector. In some embodiments, the rotary element may include another pivot point disposed between the proximal end of the extension member and the rotary element.

In a sixth set of embodiments, the rotary device may include a cutaway portion\, which may include a sector and/or a channel. In some embodiments, the extension member may be flexible and secured within the channel of the cutaway portion. In some embodiments, in response to rotation of the rotary device, the extension member may contact an edge of the sector and apply traction to the catheter assembly.

In a seventh set of embodiments, a method of applying traction to a catheter assembly may include coupling a tether device to a patient and a catheter assembly. In some embodiments, the tether device may include a base, a housing coupled to the base, a button depressible into the housing, a spring, and a tether. In some embodiments, the spring may be disposed between an inner surface of the housing and the button. In some embodiments, the spring may bias the button in a first position. In some embodiments, the button may include a hole therethrough. In some embodiments, the housing may include two other holes, which may oppose each other.

In some embodiments, the tether may extend through the hole and the two other holes. In some embodiments, in response to the button being in the first position, the tether may be locked within the tether device. In some embodiments, in response to the button being depressed from the first position to a second position, the tether may be configured to be pulled through the tether device.

In some embodiments, the method may include pressing the button and while the button is pressed, pulling the tether proximally through the tether device such that the tether between the tether device and the catheter assembly is taut. In some embodiments, the method may include releasing the button after pulling the tether proximally through the tether device such that the tether between the tether device and the catheter assembly is taut. In some embodiments, in response to releasing the button, the tether may be locked within the tether device. In some embodiments, after releasing the button, the method may include moving the base proximally. In some embodiments, in response to moving the base proximally, the tether may apply traction to the catheter assembly.

In an eighth set of embodiments, a method of applying traction to a catheter assembly may include coupling a tether device to a catheter assembly. In some embodiments, the tether device may include a base and a tether. In some embodiments, the base may include multiple holes, and a proximal end of the tether may include a peg configured to fit within the holes. In some embodiments, the method may include moving the peg from a first hole of the holes to a second hole of the holes. In some embodiments, the first hole of the holes may be proximal to the second hole of the holes. In some embodiments, in response to moving the peg from the first hole of the holes to the second hole of the holes, the tether may apply traction to the catheter assembly.

It is to be understood that both the foregoing general description and the following detailed description are examples and explanatory and are not restrictive of the invention, as claimed. It should be understood that the various embodiments are not limited to the arrangements and instrumentality illustrated in the drawings. It should also be understood that the embodiments may be combined, or that other embodiments may be utilized and that structural changes, unless so claimed, may be made without departing from the scope of the various embodiments of the present invention. The following detailed description is, therefore, not to be taken in a limiting sense.

### BRIEF DESCRIPTION OF THE DRAWINGS

Example embodiments will be described and explained with additional specificity and detail through the use of the accompanying drawings in which:

FIG. 1A is an upper perspective, exploded view of an example catheter traction system, according to some embodiments;

FIG. 1B is a side, exploded view of the catheter traction system, according to some embodiments;

FIG. 1C is an upper perspective view of the catheter traction system, according to some embodiments;

FIG. 1D is a cross-sectional view of the catheter traction system, illustrating an example peg within an example groove, according to some embodiments;

FIG. 1E is a cross-sectional view of the catheter traction system, illustrating the peg forced further into the groove, according to some embodiments;

FIG. 1F is another upper perspective, exploded view of the catheter traction system, according to some embodiments;

FIG. 1G is an upper perspective view of an example base of the catheter traction system, according to some embodiments;

FIG. 1H is a cross-sectional view of the base of FIG. 1G, according to some embodiments;

FIG. 1I is a cross-sectional view of the catheter traction system, illustrating the peg aligned with an example notch of the base of FIG. 1G, just prior to movement of the peg into the notch, according to some embodiments;

FIG. 1J is a cross-sectional view of the catheter traction system, illustrating the peg aligned with the notch of the base of FIG. 1G, after movement of the peg into the notch, according to some embodiments;

FIG. 1K is an upper perspective, exploded view of the catheter traction system, illustrating an example set of notches, according to some embodiments;

FIG. 1L is a cross-sectional view of the catheter traction system, illustrating the peg disposed within an example notch of the set of notches, according to some embodiments;

FIG. 1M is a cross-sectional view of the catheter traction system, according to some embodiments;

FIG. 1N is an upper perspective view of an example lid of the catheter traction system, illustrating example graduated markings, according to some embodiments;

FIG. 2A is a top view of the catheter traction system, according to some embodiments;

FIG. 2B is an upper perspective view of an example base of the catheter traction system, according to some embodiments;

FIG. 3A is a longitudinal cross-sectional view of another example catheter traction system, according to some embodiments;

FIG. 3B is another cross-sectional view of the catheter traction system of FIG. 3A, transverse to the cross-sectional view of FIG. 3A, according to some embodiments;

FIG. 3C is an enlarged, top view of a portion of the catheter traction system of FIG. 3A, illustrating an example tab in a first position, according to some embodiments;

FIG. 3D is an enlarged, top view of a portion of the catheter traction system of FIG. 3A, illustrating the tab in a second position, according to some embodiments;

FIG. 4A is a top view of another example catheter traction system, according to some embodiments;

FIG. 4B is an upper perspective, exploded view of the catheter traction system of FIG. 4A, illustrating an example lid, according to some embodiments;

FIG. 5A is an upper perspective view of another catheter traction system prior to coupling to an example catheter assembly, according to some embodiments;

FIG. 5B is a top view of the catheter traction system of FIG. 5A, according to some embodiments;

FIG. 5C is a cross-sectional view of an example rotary device of the catheter traction system of FIG. 5A, according to some embodiments;

FIG. 6A is a top view of another catheter traction system, according to some embodiments;

FIG. 6B is a schematic diagram of a rotary device of the catheter traction system of FIG. 6A, illustrating an example rotary element moving between preset locations, according to some embodiments;

FIG. 6C is an upper perspective view of the rotary device of FIG. 6B, according to some embodiments;

FIG. 6D is an enlarged upper perspective view of an example pivot point of the catheter traction system of FIG. 6A, according to some embodiments;

FIG. 7 is an upper perspective view of the catheter traction system of FIG. 1A, illustrating an example flexible extension member, according to some embodiments;

FIG. 8A is an upper perspective view of another example catheter traction system, illustrating an example first position of the catheter traction system, according to some embodiments;

FIG. 8B is an upper perspective view of the catheter traction system of FIG. 8A, illustrating an example tether device in an unlocked position and an example extension member moved through the tether device to achieve tautness, according to some embodiments;

FIG. 8C is an upper perspective view of the catheter traction system of FIG. 8A, illustrating the tether device in a locked position and movement of an example base in a proximal direction to apply traction to an example catheter assembly, according to some embodiments;

FIG. 8D is a cross-sectional view of the tether device, illustrating the tether device in the locked position, according to some embodiments;

FIG. 8E is a cross-sectional view of the tether device, illustrating the tether device in an unlocked position, according to some embodiments;

FIG. 9A is a top view of another catheter traction system, illustrating an example rotary device in an example first position, according to some embodiments;

FIG. 9B is a top view of the rotary device of FIG. 9B, illustrating the rotary device in an example second position, according to some embodiments;

FIG. 10 is a top view of another catheter traction system, according to some embodiments;

FIG. 11 is an upper perspective view of the catheter traction system of FIG. 1A, illustrating an example spring hose clamp, according to some embodiments;

FIG. 12A is a side view of an example connector in an example open position, according to some embodiments; and

FIG. 12B is a side view of the connector of FIG. 12B in an example closed position, according to some embodiments.

### DETAILED DESCRIPTION

In some embodiments, the catheter traction system to apply traction to an intravenous catheter may include a rotary device. In some embodiments, the catheter traction system may include an extension member, which may extend from the rotary device and may be configured to couple to a catheter assembly. In some embodiments, in response to activation of the rotary device, the extension member may be configured to apply traction to the catheter assembly.

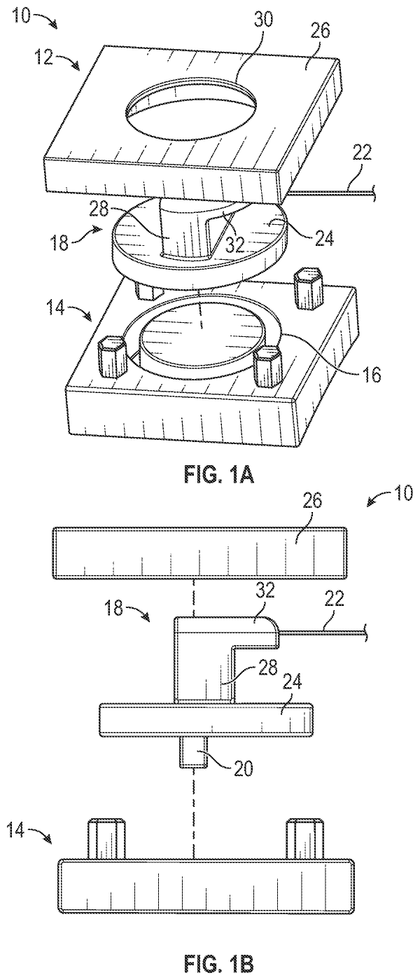

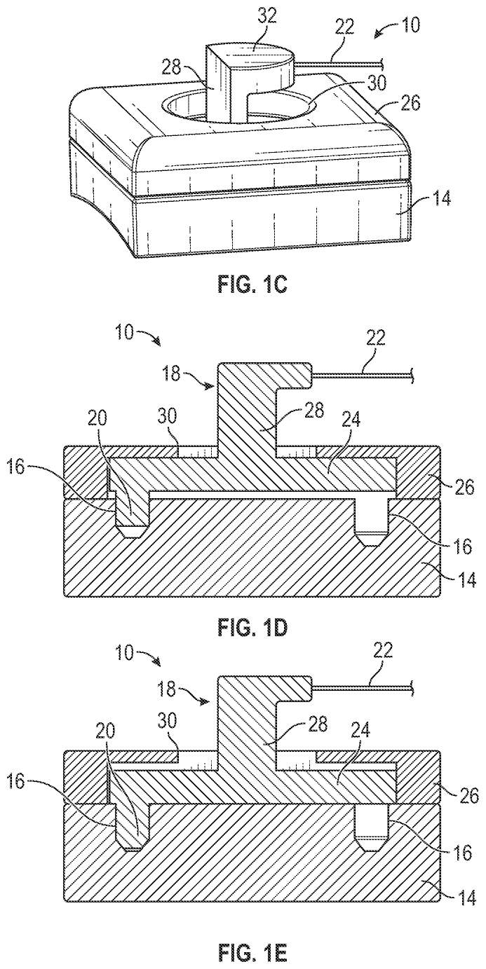

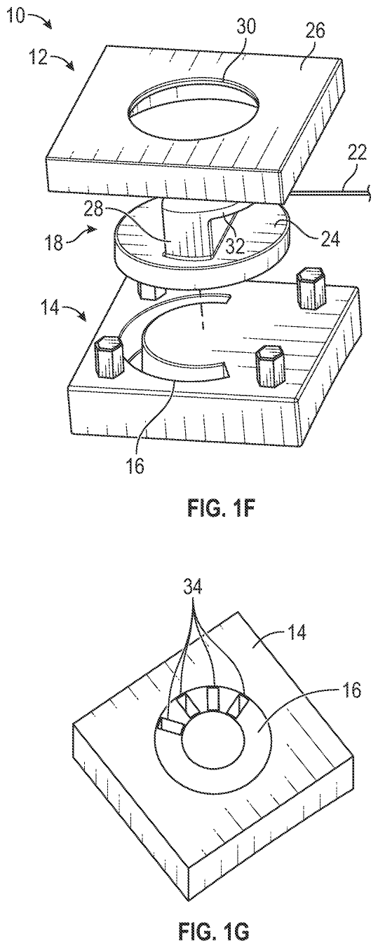

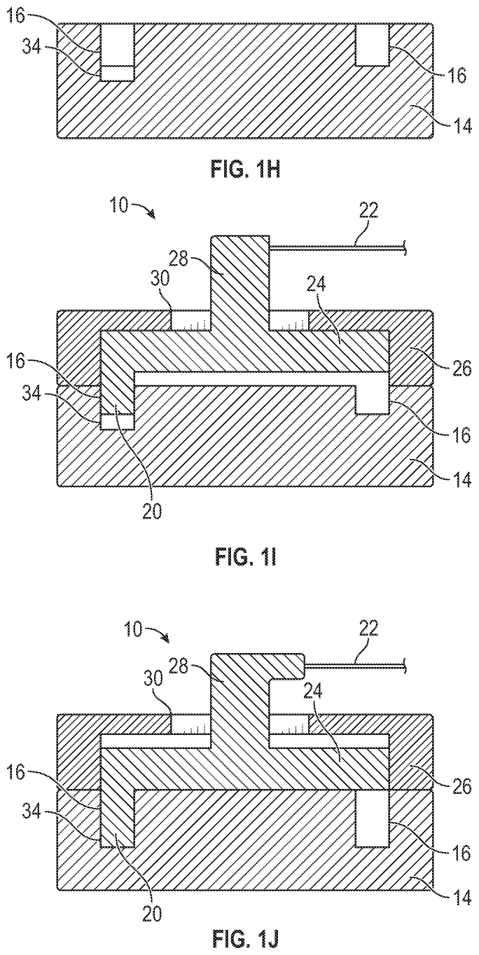

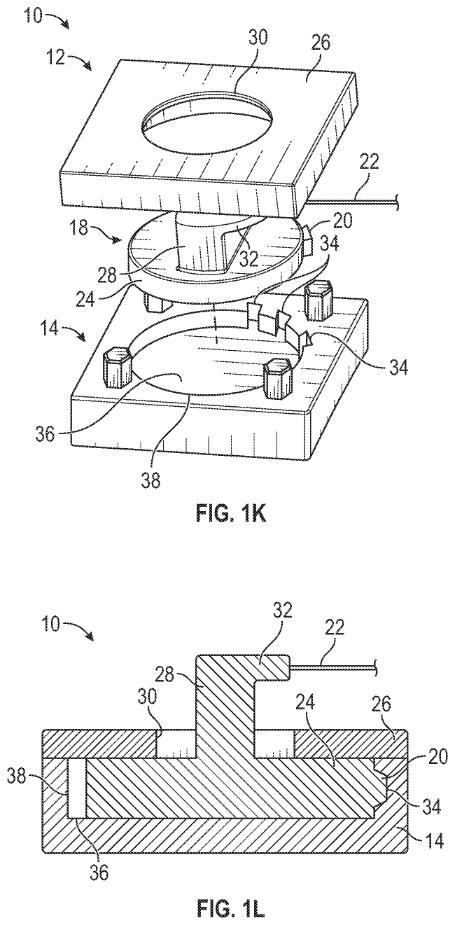

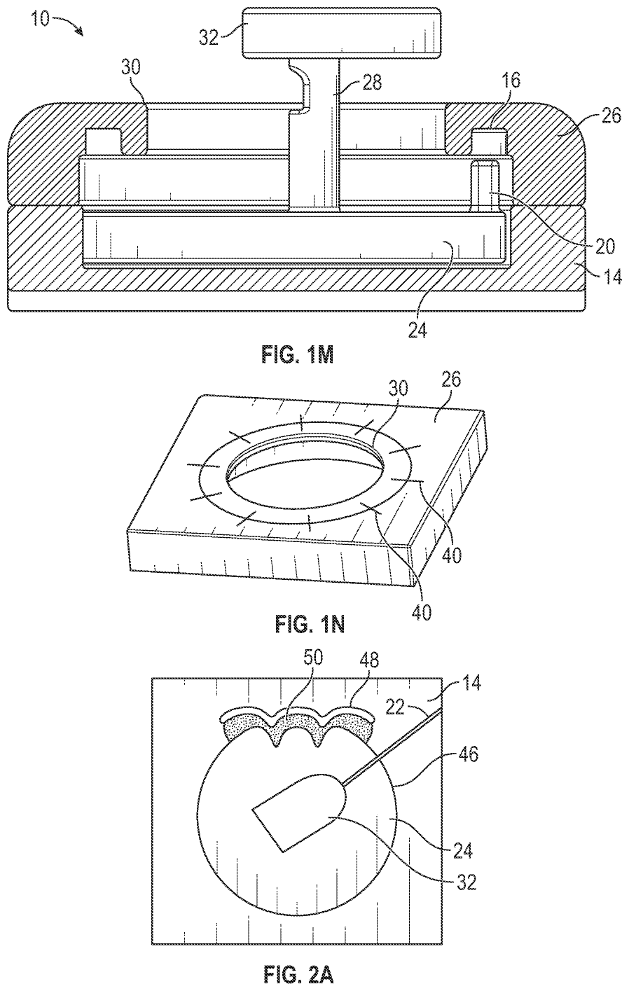

Referring now to FIGS. 1A-1E, a catheter traction system 10 may include a rotary device 12. In some embodiments, the rotary device 12 may include a base 14, a groove 16, and a rotary element 18. In some embodiments, the rotary element 18 may include a peg 20. In some embodiments, the rotary element 18 may be configured to rotate with respect to the base 14. In some embodiments, in response to the peg 20 being forced into the groove 16, rotation of the rotary element 18 with respect to the base 14 may be reduced or prevented. In some instances, when rotation is reduced, it may be more difficult for the user to accomplish or the user may need to apply increased force for rotation.

In some embodiments, in response to rotation of the rotary element 18 with respect to the base 14, an extension member 22 may be configured to apply traction to a catheter assembly. In some embodiments, the rotary element 18 may be configured to rotate clockwise and/or counterclockwise with respect to the base 14.

In some embodiments, the groove 16 may be disposed within the base 14. In some embodiments, the groove 16 may be circular, which may facilitate 360° rotation of the rotary element 18. In some embodiments, the groove 16 may include an inner circumference and an outer circumference.

In some embodiments, the peg 20 may be disposed within the groove 16. In these and other embodiments, the peg 20 may be forced further into the groove 16 to reduce or prevent rotation of the rotary element 18 with respect to the base 14. In some embodiments, the rotary element 18 may include a disc-shaped body 24. In some embodiments, a shape of the rotary element 18 may vary or may include any suitable shape. In some embodiments, the peg 20 may extend downwardly from a bottom of the disc-shaped body 24.

In some embodiments, the peg 20 may tightly fit within the groove 16. Thus, in response to the peg 20 being forced into the groove 16, the rotary element 18 may be disposed in a locked position and rotation may be prevented. In some embodiments, as illustrated, for example, in FIGS. 1D-1E, an outer portion of the groove 16 may be narrower than an inner portion of the groove 16 to create an interference fit with the peg 20 in response to the peg 20 being forced into the groove 16. In these and other embodiments, the peg 20 may be pinched or compressed when it is forced, such as pushed, into the inner portion of the groove 16. In some embodiments a width of the peg 20 may be slightly greater than or equal to the inner portion of the groove 16.

In some embodiments, the rotary device 12 may include a lid 26, which may be coupled to the base 14, and the rotary element 18 may include a neck portion 28 extending through the lid 26. In some embodiments, the lid 26 may include an opening 30 through which the neck portion 28 may extend. In some embodiments, the lid 26 may hold the peg 20 in the groove 16 and may prevent removal of the peg 20 from the groove 16, as illustrated, for example, in FIGS. 1D-1E.

In some embodiments, the lid 26 and the base 14 may be coupled together. In some embodiments, the lid 26 and the base 14 may be monolithically formed as a single unit. In some embodiments, the base 14 may include one or more pins which may extend into one or more slots of the lid 26 to facilitate securement between the lid 26 and the base 14. In some embodiments, the lid 26 may include the pins, and the base 14 may include the slots.

In some embodiments, the rotary device 12 may be secured to skin of the patient via adhesive, a bandage, tape, or any other suitable method. In some embodiments, securement of the rotary device 12 may reduce or prevent movement of the rotary device 12 with respect to the skin of the patient.

In some embodiments, the rotary element 18 may include a handle 32, which may extend outwardly from and/or perpendicular to the neck portion 28 and may facilitate gripping by a user to rotate the rotary element 18 with respect to the base 14 and/or the lid 26. In some embodiments, the extension member 22 may extend from the rotary element 18. In some embodiments, the extension member 22 may extend from the handle 32 or the neck portion 28. In some embodiments, the extension member 22 may extend from the disc-shaped body 24 and through a slot in the lid 26 (not illustrated). In some embodiments, the extension member 22 may be coupled to or monolithically formed as a single unit with the rotary element 18.

Referring now to FIG. 1F, in some embodiments, the groove 16 may be c-shaped, which may prevent the rotary element 18 from rotating a full turn. In these embodiments, the rotary element 18 may rotate less than 360°. In some embodiments, the groove 16 may include a portion of an inner circumference and a portion of an outer circumference.

Referring now to FIG. 1G-1J, in some embodiments, the groove 16 may include one or more notches 34, which may be disposed at a bottom of the groove 16. In some embodiments, in response to the peg 20 being disposed in one of the notches 34, rotation of the rotary element 18 with respect to the base 14 may be prevented or reduced. In some embodiments, the peg 20 may be automatically forced into the notches 34 when it is aligned with the notches 34, due to gravity.

Referring now to FIG. 1K-1L, in some embodiments, the rotary element 18 may include the disc-shaped body, and the peg 20 may extend outwardly from a side of the disc-shaped body. In these embodiments, the notches 34 may be disposed on a side of a circular cutout 36. In some embodiments, the peg 20 may contact an outer circumference 38 of the circular cutout 36 as the rotary element 18 rotates, and in response to the peg 20 being aligned with a particular one of the notches 34, the peg 20 may be moved by the user into the particular one of the notches 34 to lock the rotary element 18 in place.

Referring now to FIG. 1M, in some embodiments, the groove 16 may be disposed within the lid 26. In some embodiments, the rotary element 18 may include the disc-shaped body 24, and the peg 20 may extend upwardly from a top of the disc-shaped body 24. In some embodiments, the outer portion of the groove 16 may be narrower than an inner portion of the groove 16 to create an interference fit with the peg 20 in response to the peg 20 being forced into the groove 16. In some embodiments, the peg 20 may tightly fit within the groove 16 to secure the rotary element 18 in a locked position and prevent rotation. In some embodiments, the user may pull the rotary element 18 upward to force the peg 20 into the groove or further into the groove.

Referring now to FIG. 1N, in some embodiments, the lid 26 may include one or more graduated markings 40, which may indicate how much the rotary element 18 has been rotated and how much traction has been applied to the catheter assembly.

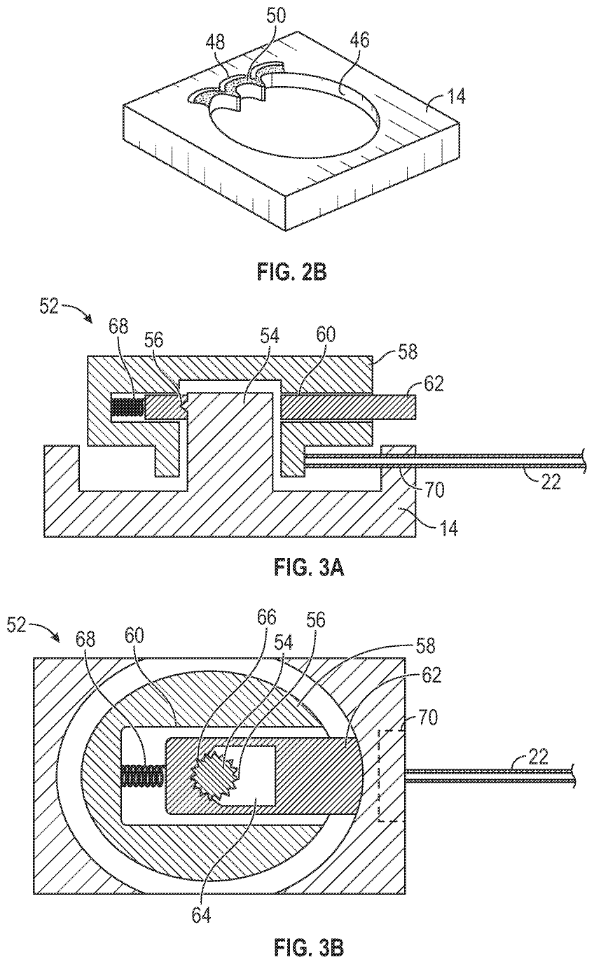

Referring now to FIGS. 2A-2B, the rotary device 12 may include the base 14, which may include an opening 46, a cutout 48, and a compliant portion 50. In some embodiments, the opening 46 may be spaced apart from the cutout by the compliant portion. In some embodiments, the compliant portion 50 may include a first shape. In some embodiments, the rotary element 18 may be disposed within the opening 30 and configured to rotate with respect to the base 14.

In some embodiments, an outer edge of the rotary element 18 may include a second shape complementary to the first shape and configured to fit into the first shape. In some embodiments, in response to the second shape being disposed within the first shape, rotation of the rotary element 18 with respect to the base 14 is reduced or prevented. In some embodiments, in response to rotation of the rotary element 18 with respect to the base 14, the extension member 22 may be configured to apply traction to the catheter assembly. In some embodiments, the first shape and the second shape may include a series of ridges. In some embodiments, a shape of the cutout 48 may correspond to the first shape, which may facilitate movement of the compliant portion 50 towards an outer edge of the cutout 48 during alignment of the first shape and the second shape.

In some embodiments, the compliant portion 50 may be constructed of a first material and a remaining portion of the base 14 may be constructed of a second material that is more rigid than the first material. In some embodiments, the compliant portion 50 may be constructed of an elastomer or another suitable material.

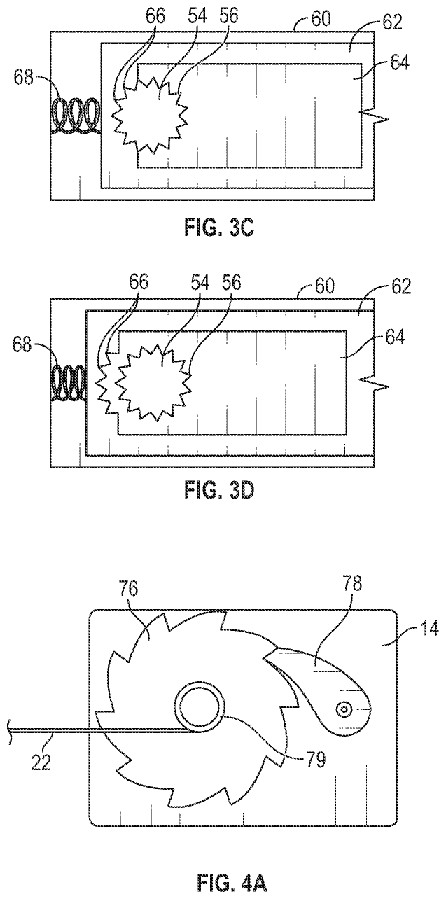

Referring now to FIGS. 3A-3D, the rotary device 52 may include the base 14, which may include a shaft 54. In some embodiments, the rotary device 52 may be similar or identical to the rotary device 12 in terms of one or more features and/or operation. In some embodiments, an outer surface of the shaft 54 may include multiple teeth 56. In some embodiments, the rotary device 52 may include a rotary element 58, which may include a slot 60. In some embodiments, the rotary device 52 may include a tab 62, which may be disposed within the slot 60 of the rotary element 58. In some embodiments, the tab 62 may include an opening 64, and the shaft 54 may extend through the opening 64. In some embodiments, an edge of the opening 64 may include multiple other teeth 66.

In some embodiments, the rotary device 52 may include a spring 68 or other biasing member, which may be disposed within the slot 60 between an inner surface of the rotary element 58 and the tab 62. In some embodiments, the spring 68 may bias the tab 62 in a first position. In some embodiments, in response to the tab 62 being in the first position, the teeth 56 may be engaged with the other teeth 66, as illustrated, for example in FIGS. 3A-3C.

In some embodiments, in response to compression of the spring 68 and the tab 62 being in a second position, the teeth 56 may not be engaged with the other teeth 66 and the rotary element 58 may be configured to rotate with respect to the base 14. In some embodiments, in response to rotation of the rotary element 58 with respect to the base 14, the extension member 22 may be configured to apply traction to the catheter assembly. In some embodiments, the extension member 22 may extend from the rotary element 58 and/or through an elongated slot 70 in the base 14, which may allow lateral movement of the extension member 22.

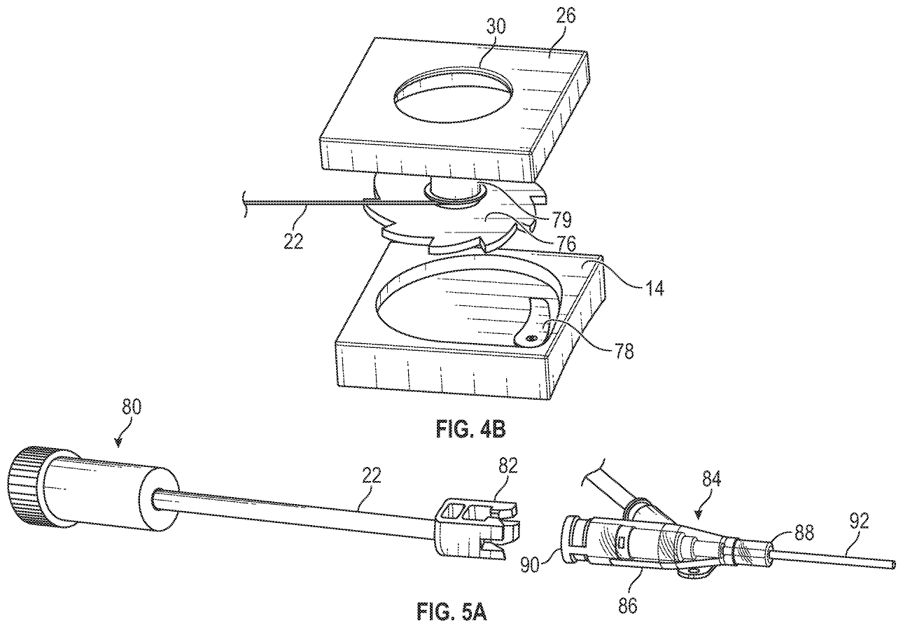

Referring now to FIGS. 4A-4B, in some embodiments, a rotary device 72 may include a ratchet 74, which may include a gear 76 and a pawl 78. In some embodiments, the rotary device 72 may be similar or identical to the rotary device 12 and/or the rotary device 52 in terms of one or more features and/or operation. In some embodiments, the extension member 22 may be coupled to the gear 76. In some embodiments, in response to rotation of the gear 76, the extension member 22 may be configured to apply traction to the catheter assembly.

In some embodiments, the gear 76 and the pawl 78 may be mounted to the base 14. In some embodiments, the rotary device 72 may include a shaft or handle 79, which may extend upwardly from the gear 76. In some embodiments, the extension member 22 may be wrapped around the handle 79.

In some embodiments, the gear 76 may be configured to rotate in a first direction to retract and/or wind the extension member 22 around the handle 79, which may apply traction to the catheter assembly. In some embodiments, the pawl 78 may be configured to engage teeth of the gear 76 to prevent rotation in a second direction opposite the first direction. In some embodiments, the pawl 78 may prevent advancement of the extension member 22 in a distal direction.

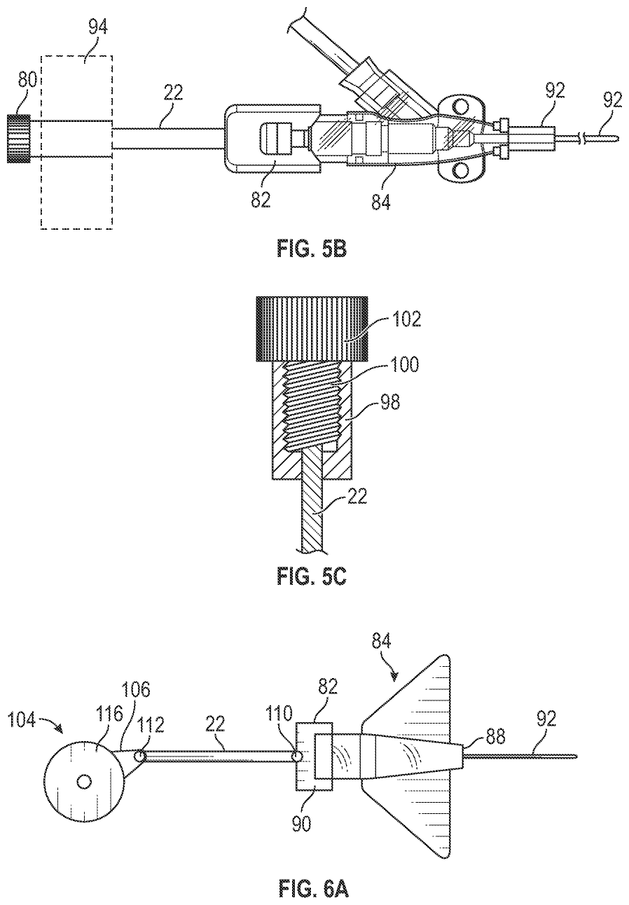

Referring now to FIGS. 5A-5B, a rotary device 80 and the extension member 22 are illustrated, according to some embodiments. In some embodiments, the rotary device 80 may be similar or identical to one or more of the following in terms of one or more features and/or operation: the rotary device 12, the rotary device 52, and the rotary device 72. As illustrated in FIGS. 5A-5C, in some embodiments, the extension member 22 may be rigid, which may facilitate application of traction to a catheter assembly 84. For example, the extension member 22 may include metal, plastic, or another suitable material. In some embodiments, a connector 82 may be disposed at a distal end of the extension member 22 and may be configured to snap onto the catheter assembly 84 or be secured to the catheter assembly 84 in another suitable manner. In some embodiments, the extension member 22 that is rigid may include or correspond to the extension member 22 in other catheter traction systems described in the present disclosure.

In some embodiments, the catheter assembly 84 may include a catheter adapter 86, which may include a distal end 88, a proximal end 90, and a lumen extending through the distal end 88 and the proximal end 90. In some embodiments, the catheter assembly 84 may include any suitable catheter assembly. In some embodiments, the catheter assembly 84 may include a catheter 92 extending from the distal end 88. In some embodiments, the catheter 92 may include a peripheral intravenous catheter, a midline catheter, or a peripherally-inserted central catheter. In some embodiments, an introducer needle (not illustrated) may extend through the catheter 92 and may be removed from the catheter assembly 84 after the catheter 92 is inserted within vasculature of a patient. In some embodiments, the connector 82 may be coupled to the proximal end 90 of the catheter adapter 86 after the introducer needle is removed from the catheter assembly 84. In some embodiments, the connector 82 may include one or more flexible arms configured to snap into one or more grooves at the proximal end 90 of the catheter adapter 86.

In some embodiments, the rotary device 80 may be secured to skin of the patient via adhesive, a bandage 94, tape, or any other suitable method. In some embodiments, securement of the rotary device 80 may reduce or prevent movement of the rotary device 80 with respect to the skin of the patient.

In some embodiments, the rotary device 80 may include a rotary element 96 coupled to a proximal end of the extension member 22. In some embodiments, the rotary element 96 may be disposed within a housing 98 and/or may be threaded to the housing 98. In some embodiments, threads 100 may be disposed on the rotary element 96, and the rotary element 96 may be unthreaded with respect to the housing 98 to apply traction to the catheter assembly 84. In some embodiments, the rotary element 96 may not include the threads 100 and may be slid proximally with respect to the housing 98 to apply traction to the catheter assembly 84. In some embodiments, a proximal end of the rotary element 96 may include a dial or handle 102, which may be rotated or pulled by the user to move the rotary element 96 with respect to the housing 98 and apply the traction.

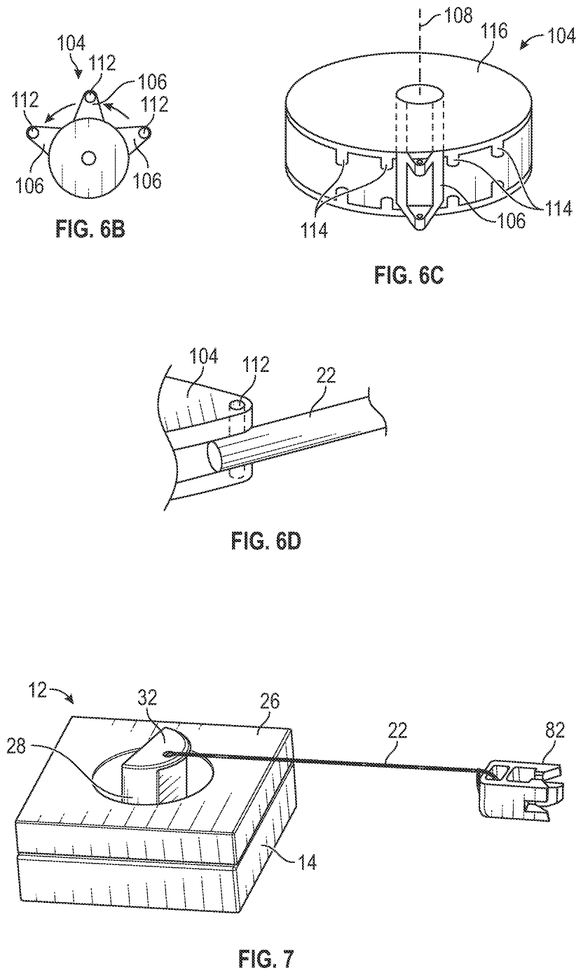

Referring now to FIGS. 6A-6D, a rotary device 104 may include a rotary element 106 configured to rotate about an axis 108 between multiple preset locations. In some embodiments, the rotary device 104 may be similar or identical to one or more of the following in terms of one or more features and/or operation: the rotary device 12, the rotary device 52, the rotary device 72, and the rotary device 80.

In some embodiments, the rotary device 104 may include a connector 82 disposed at a distal end of the extension member 22. In some embodiments, the connector 82 may be configured to couple to the catheter assembly 84. In some embodiments, the rotary element 106 may include a pivot point 110 disposed between the distal end of the extension member 22 and the connector 82. In some embodiments, the rotary element 106 may include another pivot point 112 disposed between the proximal end of the extension member 22 and the rotary element 106. In these and other embodiments, the extension member 22 may be rigid.

In some embodiments, the rotary element 106 may rotate about the axis 108 within a housing 116. In some embodiments, the preset locations may be defined by one or more catches or bumps 114, which may be disposed within the housing 116. In some embodiments, the rotary element 106 may be movable between the preset locations, but the bumps 114 may provide resistance to movement of the rotary element 106.

Referring now to FIG. 7, in some embodiments, the extension member 22 may be flexible. For example, the extension member 22 may include a string, elastic, tether, cord, or another suitable flexible member. In some embodiments, the extension member 22 may be coupled to any suitable portion of the rotary element 18, such as, for example, the handle 32. In some embodiments, the extension member 22 may be coupled to the rotary element 18 may tying, adhesive, a clasp, or any other suitable method. In some embodiments, the extension member 22 that is flexible may include or correspond to the extension member 22 in other catheter traction systems described in the present disclosure.

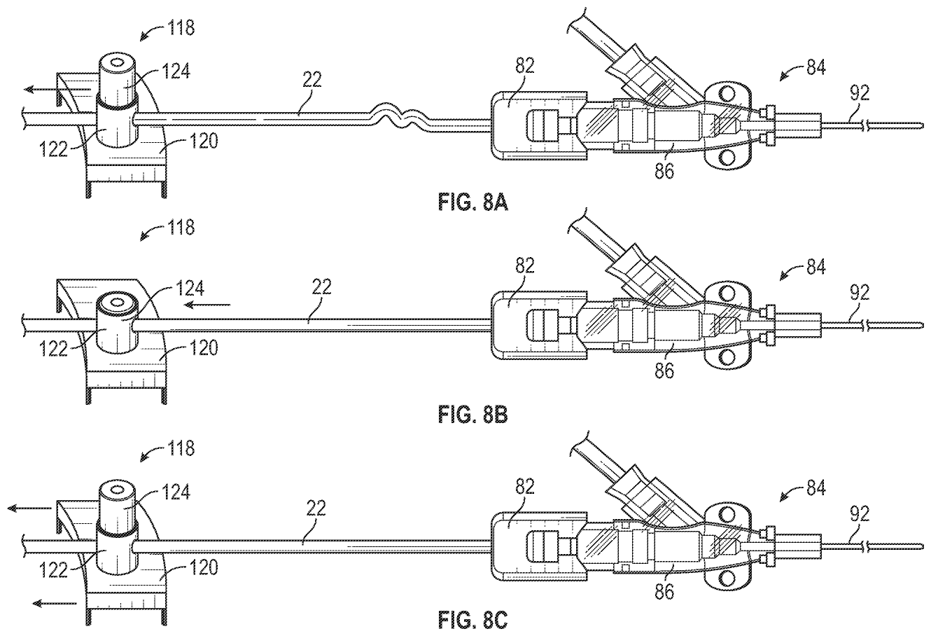

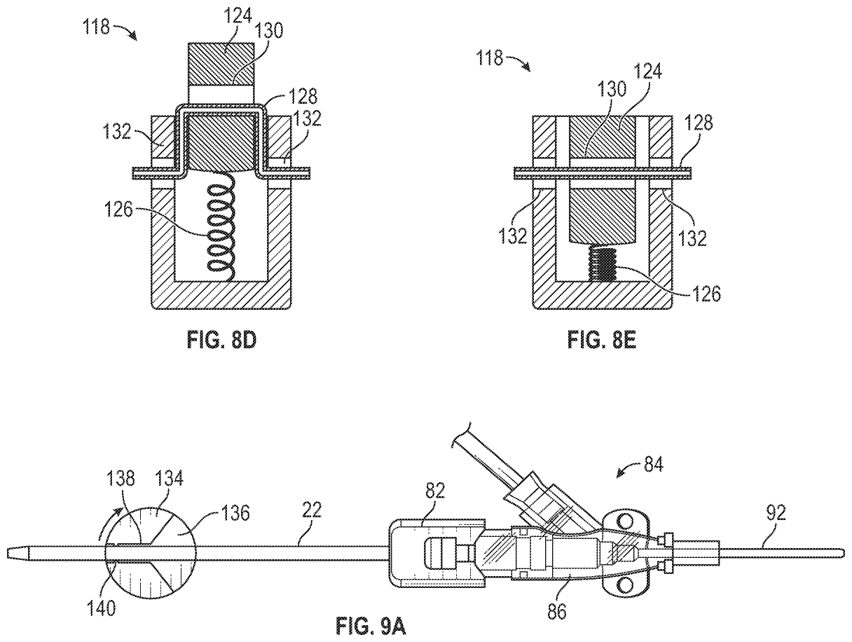

Referring now to FIGS. 8A-8E, a tether device 118 is illustrated, according to some embodiments. In some embodiments, a method of applying traction to the catheter assembly 84 may include coupling the tether device 118 to a patient and the catheter assembly 84. In some embodiments, the tether device 118 may include a base 120, a housing 122 coupled to the base 120, a button 124 depressible into the housing 122, a spring 126, and a tether 128. In some embodiments, the spring 126 may be disposed between an inner surface of the housing 122 and the button 124. In some embodiments, the spring 126 may bias the button 124 in a first position. In some embodiments, the button 124 may include a hole 130 therethrough. In some embodiments, the housing 122 may include two other holes 132, which may oppose each other. In some embodiments, the tether 128 may extend through the hole 130 and the two other holes 132.

In some embodiments, in response to the button 124 being in the first position, the tether 128 may be locked within the tether device 118, as illustrated in FIGS. 8A, 8C, and 8D. In some embodiments, in response to the button 124 being depressed from the first position to a second position, the tether 128 may be configured to be pulled through the tether device 118, as illustrated in FIGS. 8B and 8E.

In some embodiments, initially, the tether 128 may be loose and not taut, as illustrated, for example, in FIG. 8A. In some embodiments, the method may include pressing the button 124 and while the button 124 is pressed, pulling the tether 128 proximally through the tether device 118 such that the tether 128 between the tether device 118 and the catheter assembly 84 is taut, as illustrated, for example, in FIG. 8B. In some embodiments, the method may include releasing the button 124 after pulling the tether 128 proximally through the tether device 118 to achieve tautness. In some embodiments, in response to releasing the button 124, the tether 128 may be locked within the tether device 118. In some embodiments, after releasing the button 124, the method may include moving the base 120 proximally. In some embodiments, in response to moving the base 120 proximally, the tether 128 may apply traction to the catheter assembly. In some embodiments, the base 120 may be disposed on an arm band, which may be slidable in a proximal direction to apply the traction. In some embodiments, a securement, such as a bandage, tape, adhesive, etc. may be moved with the base 120 in the proximal direction or may be removed and reapplied.

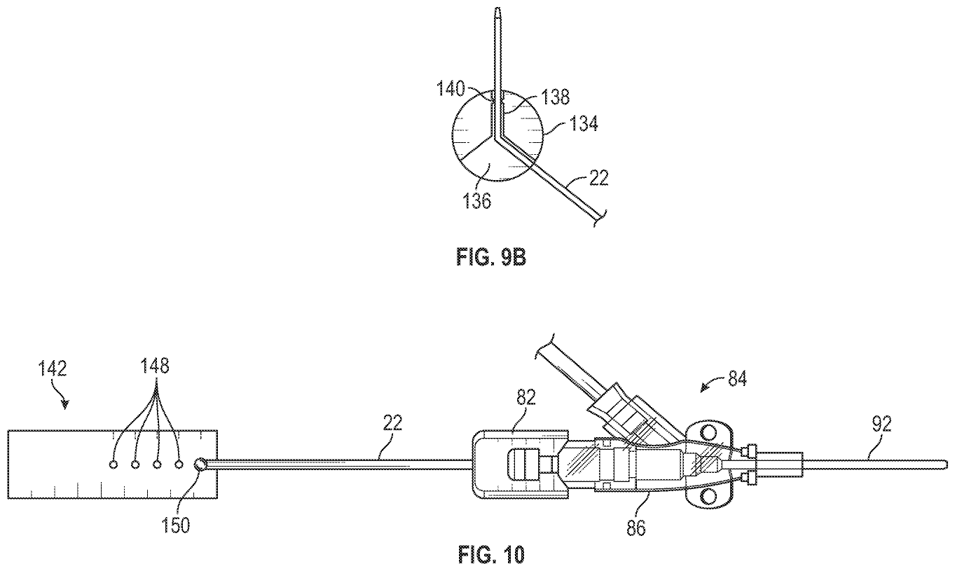

Referring now to FIGS. 9A-9B, in some embodiments, a rotary device 134 may include a cutaway portion. In some embodiments, the rotary device 134 may be similar or identical to one or more of the following in terms of one or more features and/or operation: the rotary device 12, the rotary device 52, the rotary device 72, the rotary device 80, and the rotary device 104.

In some embodiments, the cutaway portion may include a sector 136 and/or a channel 138 extending from the sector 136. In some embodiments, the extension member 22 may be flexible and secured within the channel 138 of the cutaway portion via a narrowed diameter portion 140 of the channel 138 or any other suitable method. In some embodiments, in response to rotation of the rotary device 134, the extension member 22 may contact an edge of the sector 136 and apply traction to the catheter assembly 84. In some embodiments, the rotary device 134 may be rotated from a first position, illustrated, for example, in FIG. 9A, to a second position, illustrated, for example, in FIG. 9B, to apply the traction.

Referring now to FIG. 10, a tether device 142 is illustrated, according to some embodiments. In some embodiments, a method of applying traction to the catheter assembly 84 may include coupling the tether device 142 to the catheter assembly 84. In some embodiments, the tether device 142 may include a base 144 and a tether 146. In some embodiments, the base 144 may include multiple holes 148, and a proximal end of the tether 146 may include a peg 150 configured to fit within the holes 148. In some embodiments, the method may include moving the peg 150 from a first hole of the holes 148 to a second hole of the holes 148. In some embodiments, the first hole of the holes 148 may be proximal to the second hole of the holes 148. In some embodiments, in response to moving the peg 150 from the first hole of the holes 148 to the second hole of the holes 148, the tether 146 may apply traction to the catheter assembly 84.

In some embodiments, the connector 82 may be coupled to the extension member 22 or monolithically formed with the extension member 22 as a single unit. In some embodiments, the connector 82 may include a snap connector or any suitable connector. In some embodiments, the connector 82 illustrated in FIG. 10 may be used with any of the catheter traction systems described in the present disclosure.

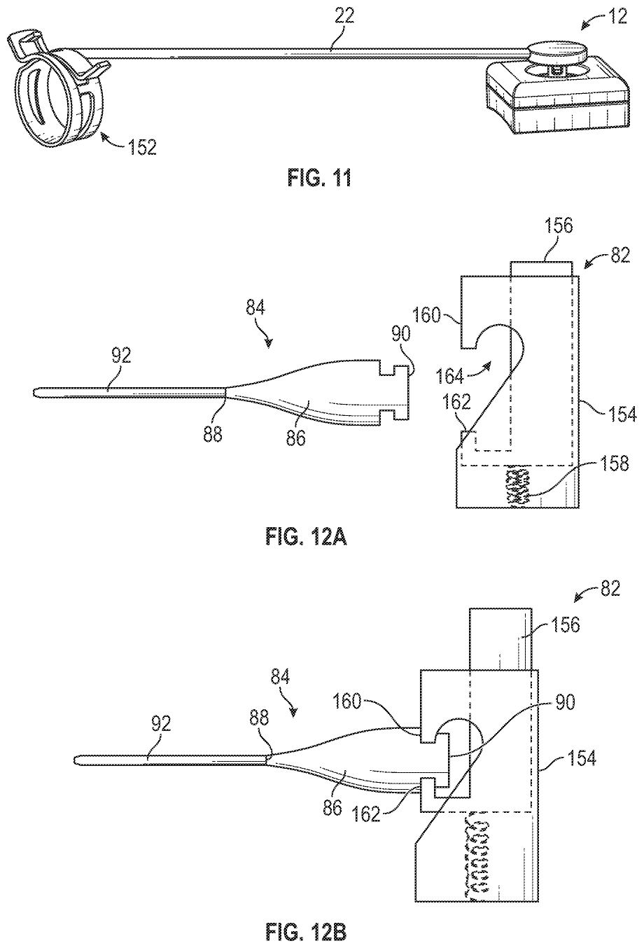

Referring to FIG. 11, in some embodiments, the connector 82 may include a spring hose clamp 152, which may be configured to clamp onto the proximal end 90 of the catheter adapter 86. In some embodiments, the connector 82 illustrated in FIG. 11 may be used with any of the catheter traction systems described in the present disclosure.

Referring now to FIGS. 12A-12B, in some embodiments, the connector 82 may include a housing 154, a button 156 depressible into the housing 154, and a spring 158 disposed between an inner surface of the housing 154 and the button 156. In some embodiments, the button 156 may include an arm 160, and the housing 154 may include another arm 162. In some embodiments, the arm 160 and the other arm 162 may form a mouth 164 configured to grasp the catheter assembly 84. In some embodiments, the connector 82 illustrated in FIG. 12 may be used with any of the catheter traction systems described in the present disclosure.

In some embodiments, the spring 158 may bias the button 156 in a first position in which the mouth 164 is closed, facilitating coupling to the catheter assembly 84, as illustrated, for example, in FIG. 12B. In some embodiments, depression of the button 156 from the first position to a second position may open the mouth 164, facilitating uncoupling of the connector 82 from the catheter assembly 84, as illustrated, for example, in FIG. 12A.

All examples and conditional language recited herein are intended for pedagogical objects to aid the reader in understanding the invention and the concepts contributed by the inventor to furthering the art and are to be construed as being without limitation to such specifically recited examples and conditions. Although embodiments of the present inventions have been described in detail, it should be understood that the various changes, substitutions, and alterations could be made hereto without departing from the spirit and scope of the invention.

1. A catheter traction system to apply traction to an intravenous catheter, the catheter traction system comprising:
a rotary device; and
an extension member extending from the rotary device and configured to couple to a catheter assembly, wherein in response to activation of the rotary device, the extension member is configured to apply traction to the catheter assembly.

2. The catheter traction system of claim 1, wherein the rotary device comprises:
a base;
a groove; and
a rotary element comprising a peg, wherein the rotary element is configured to rotate with respect to the base, wherein in response to the peg being forced into the groove, rotation of the rotary element with respect to the base is reduced, wherein in response to rotation of the rotary element with respect to the base, the extension member is configured to apply traction to the catheter assembly.

3. The catheter traction system of claim 2, wherein the groove is disposed within the base.

4. The catheter traction system of claim 2, wherein the groove is circular.

5. The catheter traction system of claim 2, wherein the groove is c-shaped.

6. The catheter traction system of claim 2, wherein the peg is disposed within the groove, wherein an outer portion of the groove is narrower than an inner portion of the groove to create an interference fit with the peg in response to the peg being forced into the groove.

7. The catheter traction system of claim 2, wherein the groove is disposed within the base, wherein the rotary element comprises a disc-shaped body, wherein the peg extends downwardly from a bottom of the disc-shaped body.

8. The catheter traction system of claim 2, wherein the rotary device further comprises a lid covering the base, wherein the groove is disposed within the lid.

9. The catheter traction system of claim 8, wherein the rotary element comprises a disc-shaped body, wherein the peg extends upwardly from a top of the disc-shaped body.

10. The catheter traction system of claim 2, wherein the rotary device further comprises a lid coupled to the base, wherein the rotary element comprises a neck portion extending through the lid, wherein the lid holds the peg in the groove.

11. The catheter traction system of claim 2, wherein the groove comprises a plurality of notches, wherein in response to the peg being forced into the plurality of notches, rotation of the rotary element with respect to the base is reduced.

12. The catheter traction system of claim 1, wherein the rotary device comprises:
a base, comprising an opening, a cutout, and a compliant portion, wherein the opening is spaced apart from the cutout by the compliant portion, wherein the compliant portion comprises a first shape; and
a rotary element disposed within the opening and configured to rotate with respect to the base, wherein an outer edge of the rotary element comprises a second shape complementary to the first shape and configured to fit into the first shape, wherein in response to the second shape being disposed within the first shape, rotation of the rotary element with respect to the base is reduced, wherein in response to rotation of the rotary element with respect to the base, the extension member is configured to apply traction to the catheter assembly.

13. The catheter traction system of claim 1, wherein the rotary device comprises:
a base, comprising a shaft, wherein an outer surface of the shaft comprises a plurality of teeth;
a rotary element, comprising a slot;
a tab disposed within the slot of the rotary element and comprising an opening, wherein the shaft extends through the opening, wherein an edge of the opening comprises a plurality of other teeth; and
a spring disposed within the slot between an inner surface of the rotary element and the tab, wherein the spring biases the tab in a first position, wherein in response to the tab being in the first position, the plurality of teeth are engaged with the plurality of other teeth, wherein in response to compression of the spring and the tab being in a second position, the plurality of teeth are not engaged with the plurality of other teeth and the rotary element is configured to rotate with respect to the base, wherein in response to rotation of the rotary element with respect to the base, the extension member is configured to apply traction to the catheter assembly.

14. The catheter traction system of claim 1, wherein the rotary device comprises:
a ratchet, comprising a gear and a pawl, wherein the extension member is coupled
to the ratchet, wherein in response to rotation of the gear, the extension member is configured to apply traction to the catheter assembly.

15. The catheter traction system of claim 14, wherein the rotary device further comprises:
a base, wherein the gear and the pawl are mounted to the base; and
a handle extending upwardly from the gear.

16. The catheter traction system of claim 15, wherein the extension member is wrapped around the handle.

17. The catheter traction system of claim 1, wherein the extension member is rigid.

18. The catheter traction system of claim 1, wherein the extension member is flexible.

19. The catheter traction system of claim 1, wherein the rotary device comprises:
a rotary element configured to rotate about an axis between a plurality of preset locations;
a connector disposed at a distal end of the extension member, wherein the connector is configured to couple to the catheter assembly;
a pivot point disposed between the distal end of the extension member and the connector; and
another pivot point disposed between the proximal end of the extension member and the rotary element.

20. The catheter traction system of claim 1, wherein the rotary device comprises a cutaway portion, wherein the cutaway portion comprises a sector and a channel, wherein the extension member is flexible and secured within the channel of the cutaway portion, wherein in response to rotation of the rotary device, the extension member contacts an edge of the sector and applies traction to the catheter assembly.

21. The catheter traction system of claim 1, further comprising a spring hose clamp disposed at a distal end of the extension member and configured to couple to the catheter assembly.

22. The catheter traction system of claim 1, further comprising a connector disposed at a distal end of the extension member, wherein the connector comprises a housing, a button depressible into the housing, and a spring disposed between an inner surface of the housing and the button, wherein the button comprises an arm and the housing comprises another arm, wherein the arm and the other arm form a mouth configured to grasp the catheter assembly, wherein the spring biases the button in a first position in which the mouth is closed, wherein depression of the button from the first position to a second position opens the mouth.

23. The catheter traction system of claim 1, further comprising a connector disposed at a distal end of the extension member and configured to snap onto the catheter assembly.

24. A method of applying traction to a catheter assembly, the method comprising:
coupling a tether device to a patient and a catheter assembly, wherein the tether device comprises:
a base;
a housing coupled to the base;
a button depressible into the housing;
a spring disposed between an inner surface of the housing and the button, wherein the spring biases the button in a first position, wherein the button comprises a hole therethrough, wherein the housing comprises two other holes; and
a tether extending through the hole and the two other holes, wherein in response to the button being in the first position, the tether is locked within the tether device, wherein in response to the button being depressed from the first position to a second position, the tether is configured to be pulled through the tether device;
pressing the button and while the button is pressed, pulling the tether proximally through the tether device such that the tether between the tether device and the catheter assembly is taut; releasing the button after pulling the tether proximally through the tether device such that the tether between the tether device and the catheter assembly is taut, wherein in response to releasing the button, the tether is locked within the tether device; and
after releasing the button, moving the base proximally, wherein in response to moving the base proximally, the tether applies traction to the catheter assembly.

25. A method of applying traction to a catheter assembly, the method comprising:
coupling a tether device to a catheter assembly, wherein the tether device comprises:
a base comprising a plurality of holes;
a tether, wherein a proximal end of the tether comprises a peg configured to fit within the plurality of holes; and
moving the peg from a first hole of the plurality of holes to a second hole of the plurality of holes, wherein the first hole of the plurality of holes is proximal to the second hole of the plurality of holes, wherein in response to moving the peg from the first hole of the plurality of holes to the second hole of the plurality of holes, the tether applies traction to the catheter assembly.
